(19) United States
(12) Patent Application Publication
(10) Pub. No.: US 2023/0042367 A1
(43) Pub. Date: Feb. 9, 2023

(54) METHODS AND COMPOSITIONS FOR TREATING CANCERS HAVING F-BOX AND WD-REPEAT PROTEIN 7 (FBXW7) ALTERATIONS AND/OR CYCLIN L1 (CCNL1) GAIN OR AMPLIFICATION

(71) Applicant: THE GOVERNING COUNCIL OF THE UNIVERSITY OF TORONTO, Toronto (CA)

(72) Inventors: Stephane Angers, Mississauga (CA); Zachary Steinhart, Toronto (CA); Siobhan O'Brien, Toronto (CA)

(21) Appl. No.: 17/782,916

(22) PCT Filed: Jul. 12, 2020
(86) PCT No.: PCT/CA2020/051683

§ 371 Date: Jun. 6, 2022

Related U.S. Application Data
(60) Provisional application No. 62/944,896, filed on Dec. 6, 2019.

(51) Int. Cl. A61K 31/4743; A61K 31/5377; C12Q 1/6872; C12Q 1/6827; G01N 33/574

(52) U.S. Cl. CPC A61K 31/4743; A61K 31/5377; C12Q 1/6872; C12Q 1/6827; G01N 33/57496; C12Q 2600/156

(57) ABSTRACT

Provided are methods of selecting a patient with cancer and/or treating a patient with cancer that comprises a deleterious alteration in FBXW7 and/or an amplification of CCNL1 for CDK11 inhibitor and/or ATR inhibitor treatment. For example, the method of selecting a patient afflicted with a cancer likely to benefit from a CDK11 inhibitor and/or ATR inhibitor treatment, the method comprising: obtaining a biological sample; testing the sample for i) loss of function FBXW7, optionally for a deleterious mutation in F box WD-repeat containing protein (FBXW7) substrate binding domain and/or ii) upregulated CCNL1, optionally a gain or amplification of CCNL1; and selecting the patient having i) loss of function FBXW7, optionally a deleterious mutation in the FBWX7 substrate-binding domain or ii) upregulated CCNL1, optionally a gain or amplification of CCNL1, as likely to benefit and/or for treatment with the CDK11 inhibitor and/or ATR inhibitor.

### RELATED FAMILY MEMBERS

This PCT Application claims the benefit of priority to U.S. Provisional Application No. 62/944,896, filed Dec. 6, 2019, which is herein incorporated by reference in its entirety.

### FIELD

The present disclosure relates to methods and compositions for identifying patients who would benefit from treatment and/or treating patients with a CDK11 inhibitor and/or an ATR inhibitor and more particularly to identifying patients having loss of function F-box and WD-repeat protein 7 (FBXW7) or upregulated Cyclin L1 (CCNL1) protein levels, optionally an amplification or gain of Cyclin L1 (CCNL1) gene, for treatment with a CDK11 inhibitor (for example OTS964) and/or an ATR inhibitor (for example AZD6738).

### BACKGROUND

Precision medicine and companion diagnostics permit stratification of patients and tailoring of treatments based on the patient's predictive response.

F-box and WD-repeat protein 7 (FBXW7) is a substrate specific receptor component of a Skp1-Cullin-Fbox (SCF) E3 ubiquitin ligase complex. FBXW7 is a well-known tumour suppressor gene; it works to control the cellular levels of a number of oncogenes, including c-Myc and Cyclin E (Yeh et al, 2018). Mutations in FBXW7 are common across cancers, and regions of mutational hotspots are located within substrate-recognition domains of the protein. Interestingly, several of these mutations appear to function in a dominant-negative manner (Yeh et al, 2016) likely reflecting the dimeric nature of functional SCF E3 ubiquitin ligases. Aberrant expression of FBXW7 in tumorigenesis has been previously described (Sailo et al., 2019).

CCNL1 (Cyclin L1) is a poorly studied cyclin. CCNL1 amplification has been identified in various cancers and in head and neck squamous cell carcinoma it was linked to worse prognosis for this cancer (Muller et al 2006). CCNL1 has been shown to act as a splicing factor, localized to splicing machinery puncta in the nucleus (Loyer et al 2008), and in association with the cyclin dependent kinase CDK11, has roles in cytokinesis (Ahmed et al 2019).

A recent study identified that a small molecule in pre-clinical development for TOPK inhibition (OTS964) may have off target activity and is a potent and selective inhibitor of CDK11 (Lin et al, 2019).

US 2016/0324878 describes combination therapies comprising OTS964 for cancer and immune diseases.

WO 2017/201528 describes prodrugs and nanoparticles for treating diseases, such as cancer, comprising OTS964.

Hu et al., 2019, has identified analogs of OTS964 having potent anti-cancer activity.

Identification of genetic interactions can lead to new precision medicine treatments and companion diagnostics which are desired.

### SUMMARY

CCNL1 is identified herein as a novel FBXW7 substrate and CDK11 is identified as a therapeutic target in this context. FBXW7^{-/-} cells are also shown herein to have a high level of phosphorylated-CHK1, a marker of DNA replication stress and activated ATR pathway. As described in the Examples, FBXW7^{-/-} cells are hypersensitive to CDK11 and ATR inhibitors, suggesting that FBXW7 mutations and/or CCNL1 amplification are biomarkers that can be used to stratify patients that could benefit from CDK11 and/or ATR inhibition. Disclosed herein are methods for personalizing cancer treatment of patients having a deleteriously altered FBXW7 and/or increased CCNL1 levels, for example due to CCNL1 chromosomal amplification or gain.

According to an aspect of the invention, there is provided a method of selecting a patient afflicted with a cancer likely to benefit from a CDK11 inhibitor and/or ATR inhibitor treatment, the method comprising:
obtaining a biological sample that was obtained from the patient;
testing the sample for i) loss of function mutation in F box WD-repeat containing protein (FBXW7), optionally a deleterious mutation in FBXW7 substrate binding domain or a deep chromosomal deletion and/or ii) upregulated CCNL1, optionally a gain or amplification of CCNL1; and
selecting the patient having i) loss of function FBXW7 mutation, optionally a deleterious mutation in the FBXW7 substrate-binding domain or ii) upregulated CCNL1, optionally amplification of CCNL1, as likely to benefit from and/or for treatment with the CDK11 inhibitor and/or the ATR inhibitor.

Also provided in another aspect, is a method of treating a patient afflicted with a cancer having i) a deleterious mutation in F box WD-repeat containing protein (FBWX7), optionally in the FBWX7 substrate binding domain or ii) upregulated CCNL1, optionally a gain or amplification of CCNL1, the method comprising administering to said patient a CDK11 inhibitor and/or an ATR inhibitor treatment.

A further aspect provides a method of treating a patient afflicted with a cancer, the method comprising:
obtaining a biological sample;
testing the biological sample for i) a deleterious mutation in F box WD-repeat containing protein (FBWX7), optionally in the FBWX7 substrate binding domain, or ii) upregulated CCNL1, optionally a gain or amplification of CCNL1; and
treating the patient having i) an alteration in F box WD-repeat containing protein (FBWX7) substrate binding domain, or ii) upregulated CCNL1, optionally a gain or amplification of CCNL1 with the CDK11 inhibitor and/or ATR inhibitor.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

An embodiment of the present disclosure will now be described in relation to the drawings in which:

FIG. 1 depicts a lollipop schematic illustrating the location of mutations in FBXW7.

FIG. 2A depicts a bar graph illustrating that the frequency of FBXW7 mutations are high across a wide range of cancers, with particular enrichment in squamous lung cancer, Esophageal cancer, head and neck cancer, uterine carcinosarcoma (endometrial) and cervical cancers. FIG. 2B depicts a bar graph illustrating cancer types and their occurrence of amplification events of CCNL1.

FIG. 3 is an oncoprint illustrating prevalence of CCNL1 and FBXW7 alterations in different cancer types, and the corresponding overall survival status of patients with these alterations.

FIG. 4 depicts the results of a genome-wide CRISPR-Cas9 dropout screen, which identifies several genes exhibiting increased growth fitness defects in FBXW7^{-/-} cells when compared to wild-type cells—including CCNL1.

FIG. 5A depicts a graph illustrating the results of a proliferation assays validating the synthetic lethality relationship between FBXW7 mutations and CCNL1 (the knockout of CCNL1 only affects the proliferation of cells harboring mutation in FBXW7 and does not affect wild-type cells (*p<0.05, ***p<0.001, one-way ANOVA). FIG. 5B depicts a line graph illustrating the results of a multicolour cell competition assay validating the synthetic lethality relationship between FBXW7 mutations and CCNL1.

FIG. 6 depicts the results of treating wild type cells and FBXW7 knock out cells with the GSK3 inhibitor CHIR99021, serum starvation, or untreated. FBXW7 knockout cells show stabilized CCNL1 levels.

FIG. 7 depicts a partial alignment of CCNL1 protein sequences across species revealing conservation of a predicted phospho-degron motif known to be targeted by phosphorylation and required for binding by a SCF E3 ubiquitin ligase. Shown are the FBXW7 CPD consensus sequence (SEQ ID NO: 8), human cyclin E sequence (SEQ ID NO: 9) human c-Myc sequence (SEQ ID NO: 10), human c-Jun sequence (SEQ ID NO: 11), CCNL1 human (SEQ ID NO: 12), CCNL1 mouse (SEQ ID NO: 13) CCNL1 rat (SEQ ID NO: 14).

FIG. 8 depicts the results of a cycloheximide chase experiment revealing the short half life of CCNL1. Overexpression of wild-type FBXW7 further shorten the half life, whereas expression of the FBXW7 R505C mutant frequently found in cancers, leads to CCNL1 stabilization. Mutation of the predicted phospho-degron motif leads to CCNL1 stabilization.

FIG. 9 depicts a line graph that illustrates the proliferation response of wild type cells and FBXW7 knock out cells when exposed to the CKD11 inhibitor OTS964. The FBXW7 knockout cells are hypersensitive to the CDK11 inhibitor.

FIG. 10 depicts a panel of images illustrating cervical organoids treated with dose varying concentrations of OTS964 for 7 days compared to an untreated control.

FIG. 11 depicts a bar graph illustrating the relative viability of patient-derived cervical cancer organoids during a 7-day treatment with varying concentrations of OTS-964 compared to an untreated control.

FIG. 12 is a series of images that depict the results of Western-blotting of organoid lysates illustrating the expression levels of CCNL1 and GAPDH in different cervical cancer organoid models. Short and long refer to the length of exposure for the detection.

FIG. 13 depicts Sanger sequencing traces revealing the presence of FBXW7 mutations in the cervical organoid Model 4 that exhibits hypersensitivity to the CDK11 inhibitor OTS964. Model 4 contains a R505L mutation. The top sequence (SEQ ID NO: 15) is the sequence of the sequencing run showing the heterozygous mutation in FBXW7 in the sequencing trace for model 4. The sequences directly below the trace show a portion of the reference nucleotide sequence of FBXW7 (SEQ ID NO: 16) aligned with the corresponding amino acid sequence (SEQ ID NO: 17). The reference nucleotide sequence of FBXW7 (SEQ ID NO: 18) is aligned to the model 4 sequence demonstrating that the mutation is at amino acid arginine 505.

FIG. 14A depicts a line graph illustrating the OTS964 dose response in a panel of cervical cancer cell lines Ca-Ski, DoTc2-4510, SiHa, C-33-A. Only the C-33-A line is hypersensitive to the CDK11 inhibitor OTS964. C-33-A cells harbor a FBXW7 mutation and express high levels of CCNL1. FIG. 14B depicts an image illustrating the cellular levels of CCNL1 and GAPDH in Ca-Ski, DoTc2-4510, SiHa, C-33-A cell lines.

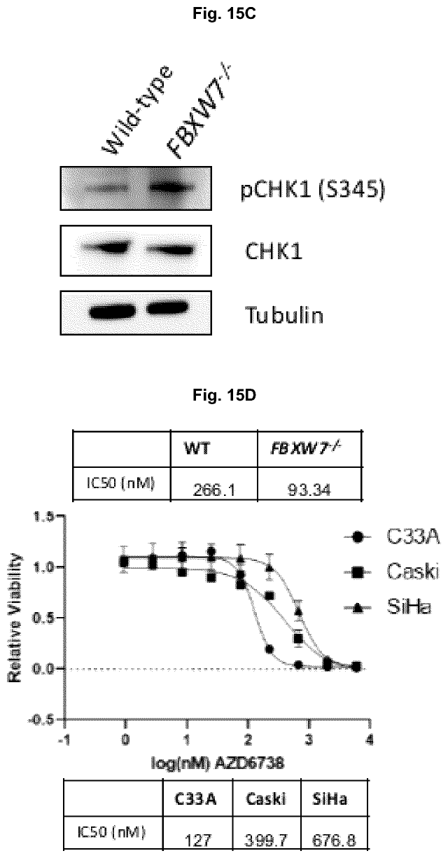

FIG. 15A depicts the results of a genome-wide CRISPR screen performed in isogenic wild-type and FBXW7^{-/-} HPAF cell lines. The results highlight several genes composing a signature of DNA replication stress that display increased growth fitness defects in the FBXW7^{-/-} cell line when compared with the wild type cells. FIG. 15B depicts the results of a cell viability assay where HPAF-II wildtype and FBXW7^{-/-} cells were treated with varying doses of the ATR inhibitor AZD6738, with the FBXW7^{-/-} exhibiting hypersensitivity to cell death when treated with this compound. FIG. 15C depicts a western blot wherein FBXW7^{-/-} cells show a high level of phosphorylated-CHK1, a marker of DNA replication stress and activated ATR pathway. FIG. 15D depicts a cell viability assay using a cervical cancer cell line panel and ATR inhibitor AZD6738 where the C33A line harbors an FBXW7 mutation and is highly susceptible to AZD6738 induced cell death. FIG. 15E depicts HPAF-II wildtype and FBXW7^{-/-} cells under confocal microscopy, where three markers of active DNA damage (as a result of replication stress) were assessed—53BP1, gH2AX, and RPA32. In all cases, FBXW7^{-/-} cells show increased expression of these markers. FIG. 15F depicts dot plots of the quantification of confocal microscopy in 15E.

### DETAILED DESCRIPTION OF THE DISCLOSURE

Unless otherwise defined, scientific and technical terms used in connection with the present disclosure shall have the meanings that are commonly understood by those of ordinary skill in the art. Further, unless otherwise required by context, singular terms shall include pluralities and plural terms shall include the singular. For example, the term “a cell” includes a single cell as well as a plurality or population of cells. Generally, nomenclatures utilized in connection with, and techniques of, cell and tissue culture, molecular biology, and protein and oligonucleotide or polynucleotide chemistry and hybridization described herein are those well-known and commonly used in the art (see, e.g. Green and Sambrook, 2012).

### Definitions

As used herein a “CDK11 inhibitor” means any molecule including a compound (e.g. small molecule drug such as OTS964), protein, such as an antibody, genetic inhibitor (siRNA, antisense etc.) that is capable of inhibiting the expression and/or protein kinase activity of a CDK11 (e.g. CDK11b) for example by at least 80%, at least 90%, or at least 95%, under for example standard CDK11 kinase conditions.

As used herein, “CDK11” refers to cyclin-dependent kinases including CDK11A (Gene ID: 728642) and/or CDK11B (Gene ID: 984).

As used herein “OTS964” means a molecule that inhibits CDK11, is formally named 9-[4-[(1R)-2-(dimethylamino)-1-methylethyl]phenyl]-8-hydroxy-6-methyl-thieno[2,3-c]quinolin-4(5H)-one and has the following chemical structure:

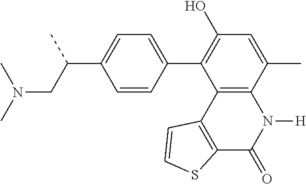 [SMILES: Cc1cc(O)c(-c2ccc([C@@H](C)CN(C)C)cc2)c2c1[nH]c(=O)c1sccc12]

as well as pharmaceutically acceptable salts thereof.

As used herein, “ATR” refers to Ataxia telangiectasia and Rad3 related kinase the human sequence of which is found in accession number Q13535.

As used herein, “ATR” inhibitor” means any molecule including a compound, protein, such as an antibody, genetic inhibitor that is capable of inhibiting the expression and/or protein kinase activity of ATR for example by at least 80%, at least 90%, or at least 95%, under for example standard ATR kinase conditions. For example, ATR inhibitors are described in U.S. Pat. Nos. 10,392,376, 10,800,769 and 10,800,774 (titled Heterocyclic inhibitors of ATR kinase), U.S. Pat. No. 10,421,765, (titled Tetrahydropyrido[4,3-d]pyrimidine inhibitors of ATR kinase), U.S. Pat. No. 10,301,324 (titled Ataxia telengiectasia and rad3-related (ATR) inhibitors and methods of their use), each of which are incorporated by reference in their entirety. AZD6738 is an example of an ATR inhibitor as demonstrated herein. ATR inhibitors that can be used include for example, BAY1895344, berzosertib, RP-3500 as well as ATR inhibitors which are in preclinical development such as AZ20(AZD6738 analogue) (Foote, K M et al, 2013; Foote, K M et al 2018), VE-821 and VE-822 (also known as VX-970) (Vertex), and ETP46464 (Toledo, L. I. et al, 2011) as well as combinations thereof.

As used herein, “AZD6738”, a molecule that inhibits ATR, refers to (R)-imino(methyl)(1-(6-((R)-3-methylmorpholino)-2-(1H-pyrrolo[2,3-b]pyridin-4-yl)pyrimidin-4-yl)cyclopropyl)-I6-sulfanone, is also known as Ceralasertib, and has the following chemical structure:

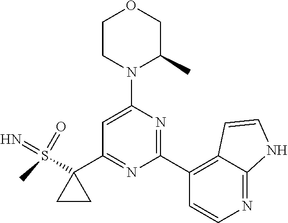 [SMILES: C[C@@H]1COCCN1c1cc(C2([S@](C)(=N)=O)CC2)nc(-c2ccnc3[nH]ccc23)n1]

as well as pharmaceutically acceptable salts thereof.

As used herein “a biological sample” means any sample from a subject such as a human and comprises depending on the assay cancer cells or suspected cancer cells or a fraction thereof, for example tumor cells, including for example a tumor tissue sample such as a biopsy, tissue slice, or cancer cells, such as blood cancers or circulatory or circulating tumor cells, which can be obtained by liquid biopsy, and including circulating DNA, etc. Fractions thereof include for example, nucleic acid fractions, (e.g. genomic DNA or mRNA) for testing for genetic alterations or protein fractions, for example when assessing levels of CCNL1. The biological sample can be a tumour biopsy, scraping, fluid such as blood, or fraction of any of the foregoing.

As used herein “analog of OTS964” includes structurally related molecules such as for example, (R)-1-(4-(1-aminopropan-2-yl)phenyl)-2-hydroxy-4-methylphenanthridin-6(5H)-one hydrochloride or (R)-1-(4-(1-aminopropan-2-yl)phenyl)-2,7-dihydroxy-4-methylphenanthridin-6(5H)-one hydrochloride.

As used herein “analog of AZD6738” includes structurally related molecules. For example analogs of AZD6738 are shown in Foote et al, J Med Chem 2018, 61, 22, 9889-9907 available at https://pubs.acs.org/doi/10.1021/acs.jmedchem.8b01187 and herein incorporated by reference. In particular, the analog of AZD6738 includes AZ20.

As used herein, the term “likely to benefit” refers to the probability, as deemed for example by the person skilled in the art, that a particular treatment will have positive effects on a patient. For example, a patient having a tumor with a deleterious mutation in the FBXW7 substrate binding domain (impairing CCNL1 binding) or an amplification of CCNL1, is more likely to respond to a CDK11 inhibitor and/or an ATR inhibitor than someone without the mutation or amplification.

The term “biomarker”, as used herewith, refers to a measurable substance or alteration in a substance for example a deleterious genetic alteration such as a deleterious mutation, or gain or amplification, which is characteristic for a specific situation, for example, associated with increased sensitivity to a treatment of inhibitor.

As used herein, the terms “patient” or “subject” may be used interchangeably herein, and refer to a mammalian subject, and preferably to a human.

The terms “treat” or “treating”, as used herein, unless otherwise indicated, mean reversing, alleviating, inhibiting the progression of, or preventing the disorder or condition to which such term applies, or one or more symptoms of such disorder or condition. The term “treatment”, as used herein, unless otherwise indicated, refers to the act of treating, as defined immediately above.

The term “effective amount” as used herein is an amount of an inhibitor that is sufficient to reduce cell growth or proliferation of cells, and/or which alleviates at least one symptom as found for the disease to be treated. Alleviating is meant to include, e.g., treating, reducing the symptoms of, or curing the disease or condition.

“Tumor”, as used herein, refers to all neoplastic masses of tissue, and all pre-cancerous and cancerous tissue growths.

The terms “cancer” and “cancerous” refer to any malignant and/or invasive proliferation, growth or tumor caused by abnormal cell growth. As used herein “cancer” includes solid tumors named for the type of cells that form them, cancer of blood, bone marrow, or the lymphatic system. The term “cancer” includes, but is not limited to, a primary cancer that originates at a specific site in the body, a metastatic cancer that has spread from the place in which it started to other parts of the body, a recurrence from the original primary cancer after remission, and a second primary cancer that is a new primary cancer in a person with a history of previous cancer of different type from latter one. The term “cancer” includes for example the cancers listed in Tables 2 and/or 3.

As used herein, the term “cancer cells” refer to cells of a cancer, e.g. cells that acquire a characteristic set of functional capabilities during their development, including the ability to evade apoptosis, self-sufficiency in growth signals, insensitivity to anti-growth signals, tissue invasion/metastasis, significant growth potential, and/or sustained angiogenesis.

The term “afflicted” includes subjects suffering from the disease and/or diagnosed with the disease.

The term “select” with respect to selecting a patient, for example for treatment, to be engaged for testing for personalized medicine, or participate in a clinical trial refers to a physical selection, for example, a medical professional or allied health worker providing an indication of the selection, for example in written form, computer communication, or verbal communication.

The term “deleterious alteration” as used herein is meant to refer to a deleterious change in a gene or level transcript/protein. As used herein, such deleterious alterations include gains and amplifications which for example lead to increased levels and/or activity (e.g. as in the case of CCNL1), and deleterious mutations such as deletion mutations, deep chromosomal deletions, point mutations, insertion mutations, or missense mutations, including those that cause protein truncation, that result in loss of function of the gene product (such as in the case of FBXW7). Deep deletion refers to a homozygous loss (e.g. both alleles for a given gene have been mutated) and shallow deletions refer to a heterozygous loss (e.g. one copy is mutated/deleted).

As used herein with reference to a gene abnormality, the terms “amplification” or “gain” refers to the presence of a higher than normal number of copies of a genomic nucleic acid sequence. “Amplification” refers to multiple copy gain and typically very large copy numbers and “gain” refers to greater than 2 copies and typically few or low level copy gain.

The term “cancer of squamous origin” as used herein, means a cancer of a squamous cell origin. Examples of cancers of a squamous origin include, cervical squamous cell carcinoma, head and neck squamous cell carcinoma, esophageal squamous cell carcinoma, squamous cell lung cancer.

The term “biopsy” as used in the description of the invention includes all types of biopsies known to those skilled in the art. Thus, the term “biopsy”, as used in the context of the present invention, may include, for example, samples obtained by resection of tumors, tissue samples obtained by endoscopic methods, or organ biopsies obtained using forceps or a needle, or liquid biopsy such as circulating tumor cells or circulating tumor DNA.

As used herein “wild-type” refers to the naturally occurring sequence of a nucleic acid at a genetic locus in the genome of an organism, or a sequence transcribed or translated from such a nucleic acid.

The term “PCR” as used herein refers to polymerase chain reaction and includes any method, including those that rely on thermal cycling.

The term “assay” as used herein refers to a procedure used for the quantitative or qualitative analysis of an analyte.

As used herein, the term “standard polypeptide assay” refers to assays used to detect or measure a level of a polypeptide. Many such assays are known to those skilled in the art, including, for example, Western blots, immunoblots, enzyme-linked immunosorbent assays (ELISAs), including competitive ELISAs, radioimmunoassay (RIA), surface plasmon resonance, fluorescence activated cell sorting (FACS), and flow cytometry.

As used herein, the term “point mutation” refers to the existence a nucleotide change (i.e. mutation) at a site relative to a wildtype sequence and/or the identity of the nucleotide present at the site of the mutation in the mutant copy of a genomic locus. The nucleotide can be in any chain of a double stranded DNA molecule.

As used herein, the term “truncation” refers to a shortening in the amino acid sequence of protein. A protein truncation may be the result of a truncation in the nucleic acid sequence encoding the protein, a substitution or other mutation that creates a premature stop codon without shortening the nucleic acid sequence, or from alternate splicing of RNA in which a substitution or other mutation that does not itself cause a truncation results in aberrant RNA processing.

The term “deletion” in the context of deletion mutants as used herein, refers to the removal or loss of one or more nucleotides from a nucleic acid sequence and includes for example deep chromosomal deletions.

The term “FBXW7” refers to F-box/WD repeat-containing protein 7 protein as well as the FBXW7 gene (Gene ID: 55294; also known as F-Box Protein FBX30, SEL-10, HCdc4, FBW7, and HAgo), depending on the context. FBXW7 is a member of the F-box protein family, which is characterized by an approximately 40 amino acid motif, the F-box and 7 tandem WD repeats. The substrate binding domain of FBXW7 lays within the WD repeats, spanning amino acids 324-659.

As used herein, the term “loss of function FBXW7” refers to a FBXW7 protein that lacks or has decreased biological function, such as or specifically its substrate binding ability or that prevents or impairs ubiquitination of a bound substrate. Loss of function FBXW7 can contain for example a deleterious mutation in its substrate-binding domain that results in the loss of function, for example, the loss of binding to substrate CCNL1.

As used in this specification and the appended claims, the singular forms “a”, “an” and “the” include plural references unless the content clearly dictates otherwise. Thus for example, a composition containing “a compound” includes a mixture of two or more compounds. It should also be noted that the term “or” is generally employed in its sense including “and/or” unless the content clearly dictates otherwise.

As used in this application and claim(s), the word “consisting” and its derivatives, are intended to be close ended terms that specify the presence of stated features, elements, components, groups, integers, and/or steps, and also exclude the presence of other unstated features, elements, components, groups, integers and/or steps.

The terms “about”, “substantially” and “approximately” as used herein mean a reasonable amount of deviation of the modified term such that the end result is not significantly changed. These terms of degree should be construed as including a deviation of at least ±5% or at least ±10% of the modified term if this deviation would not negate the meaning of the word it modifies.

The definitions and embodiments described in particular sections are intended to be applicable to other embodiments herein described for which they are suitable as would be understood by a person skilled in the art. For example, in the following passages, different aspects are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

### Methods and Kits

It is demonstrated herein that Cyclin L1 (CCLN1) is synthetic lethal with mutations within the tumor suppressor F box WD-repeat containing protein (FBXW7) and that knock down and/or mutation in FBXW7 results in increased levels of CCLN1. It is further demonstrated, that a CDK11 inhibitor or an ATR inhibited proliferation of cells having a deleterious mutation in FBXW7 and/or upregulated CCLN1, which occurs for example in cancers where CCLN1 is amplified.

Accordingly, a first aspect includes a method of selecting a patient afflicted with a cancer likely to benefit from a CDK11 inhibitor and/or an ATR inhibitor treatment, the method comprising:
testing a biological sample previously obtained from the patient for i) loss of function FBXW7 mutation, optionally for a deleterious mutation in F box WD-repeat containing protein (FBXW7) substrate binding domain and/or ii) upregulated CCNL1, optionally a gain or amplification of CCNL1;
selecting the patient having i) loss of function FBXW7 mutation, optionally a deleterious mutation in the FBWX7 substrate-binding domain and/or ii) upregulated CCNL1, optionally a gain or amplification of CCNL1 as likely to benefit and/or for treatment with the CDK11 inhibitor and/or an ATR inhibitor.

Also provided in another aspect, is a method of treating a patient afflicted with a cancer having i) a deleterious mutation in F box WD-repeat containing protein (FBXW7), optionally in the FBWX7 substrate binding domain or ii) upregulated CCNL1, optionally a gain or amplification of CCNL1, the method comprising administering to said patient a CDK11 inhibitor and/or an ATR inhibitor treatment.

A further aspect provides a method of treating a patient afflicted with a cancer, the method comprising:

testing a biological sample obtained from the patient for i) a deleterious mutation in F box WD-repeat containing protein (FBWX7), optionally in the FBWX7 substrate binding domain or ii) upregulated CCNL1, optionally a gain or amplification of CCNL1;

treating the patient having i) an alteration in F box WD-repeat containing protein (FBWX7) substrate binding domain, or ii) upregulated CCNL1, optionally a gain or amplification of CCNL1 with the CDK11 inhibitor and/or the ATR inhibitor.

Some methods comprise obtaining a biological sample. For example, the biological sample can be obtained previously from the patient. The biological sample can be a biopsy, a blood sample, or any other sample that may comprises cancer DNA that can be assayed. For example, the biological sample can be processed using one or more steps as described in the Examples.

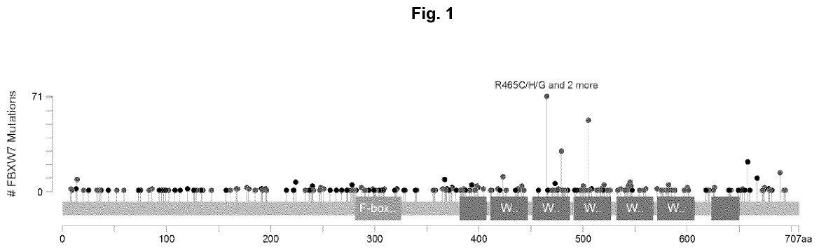

As is demonstrated for example in FIGS. 1, 2 and 3, and Tables 2 and 3, a variety of cancers have been shown to have a loss of function mutation within FBXW7 or a gain or amplification in CCNL1.

In another aspect, the cancer is selected from a cancer listed in Table 2 and Table 3 or any cancer type reported to have a FBXW7 deleterious mutation or upregulated CCNL1, for example due to gene amplification or gain. In a further embodiment, the cancer is a squamous origin cancer. In another embodiment, the cancer is cervical cancer, endometrial cancer, and/or head and neck cancer. Other embodiments, include other combinations of cancers from Table 2 and/or 3. For example, the cancers can be any sub-combinations of the cancers listed in Table 2 and/or 3 for example 1, 2, 3 or more of the cancers listed in Table 2 and/or 3.

In one embodiment, the cancer is an Endometrial Carcinoma. In one embodiment, the cancer is a Colorectal Adenocarcinoma, In one embodiment, the cancer is a Cervical Squamous Cell Carcinoma. In one embodiment, the cancer is a Cervical Adenocarcinoma. In one embodiment, the cancer is an Esophagogastric Adenocarcinoma. In one embodiment, the cancer is a Bladder Urothelial Carcinoma. In one embodiment, the cancer is a Head and Neck Squamous Cell Carcinoma. In one embodiment, the cancer is an Undifferentiated Stomach Adenocarcinoma. In one embodiment, the cancer is an Esophageal Squamous Cell Carcinoma. In one embodiment, the cancer is a Melanoma. In one embodiment, the cancer is a Non-Small Cell Lung Cancer. In one embodiment, the cancer is a Pancreatic Adenocarcinoma. In one embodiment, the cancer is a Sarcoma. In one embodiment, the cancer is an Invasive Breast Carcinoma. In one embodiment, the cancer is an Ovarian Epithelial Tumor. In one embodiment, the cancer is a Diffuse Glioma. In one embodiment, the cancer is a Mature B-Cell Neoplasm. In one embodiment, the cancer is a Pheochromocytoma. In one embodiment, the cancer is a Hepatocellular Carcinoma. In one embodiment, the cancer is an Ocular Melanoma. In one embodiment, the cancer is a Pleural Mesothelioma. In one embodiment, the cancer is a Glioblastoma. In one embodiment, the cancer is a Prostate Adenocarcinoma. In one embodiment, the cancer is a Well-Differentiated Thyroid Cancer. In one embodiment, the cancer is a Renal Clear Cell Carcinoma. In one embodiment, the cancer is a Renal Non-Clear Cell Carcinoma. In one embodiment, the cancer is an Adrenocortical Carcinoma. In one embodiment, the cancer is a Cholangiocarcinoma. In one embodiment, the cancer is a Leukemia Miscellaneous Neuroepithelial Tumor. In one embodiment, the cancer is a Seminoma. In one embodiment, the cancer is a Non-Seminomatous Germ Cell Tumor. In one embodiment, the cancer is a Thymic Epithelial Tumor. In one embodiment, the cancer is a Seminoma Invasive Breast Carcinoma.

In an embodiment, the cancer is a cervical cancer or an endometrial cancer. In yet another embodiment, the cancer is a uterine carcinosarcoma.

In some embodiments the patient is treated with a treatment that includes a CDK11 inhibitor and optionally one or more other agents, such as one or more chemotherapeutics. OTS964 has recently been shown to inhibit CDK11.

In some embodiments, the treatment comprises one or more standard of care treatments. For example, the treatment can comprise one or more of gemcitabine olaparib, paclitaxel, carboplatin, acalabrutinib, durvalumab and cisplastin. The treatment can for example comprise OTS964 and gemcitabine olaparib, paclitaxel, carboplatin, acalabrutinib, durvalumab and/or cisplastin.

In some embodiments, the CDK11 inhibitor is selected from OTS964 and analogs thereof, optionally, (R)-1-(4-(1-aminopropan-2-yl)phenyl)-2-hydroxy-4-methylphenanthridin-6(5H)-one hydrochloride or (R)-1-(4-(1-aminopropan-2-yl)phenyl)-2,7-dihydroxy-4-methylphenanthridin-6(5H)-one hydrochloride. In one embodiment, the CDK11 inhibitor is OTS964.

The CDK11 inhibitor can for example be formulated for oral delivery or formulated in a liposomal formulation for intravenous delivery.

Several formulations have been tested in animals, for example OTS964 has been delivered orally in a PBS+ glucose solution at 50-100 mg/kg. Liposomal OTS964 formulations have also been used in animals for example delivered intravenously at 40-60 mg/kg.

In one embodiment, the ATR inhibitor is selected from ATR inhibitors described in U.S. Pat. Nos. 10,392,376, 10,800,769 and 10,800,774 (titled Heterocyclic inhibitors of ATR kinase), U.S. Pat. No. 10,421,765, (titled Tetrahydropyrido[4,3-d]pyrimidine inhibitors of ATR kinase), or U.S. Pat. No. 10,301,324 (titled Ataxia telengiectasia and rad3-related (ATR) inhibitors and methods of their use), each of which are incorporated by reference in their entirety. In one embodiment, the ATR inhibitor is selected from AZD6738, BAY1895344, berzosertib, RP-3500 AZ20(AZD6738 analogue), VE-821 and VE-822 (also known as VX-970) (Vertex), and ETP46464 as well as combinations thereof.

In another embodiment, the ATR inhibitor comprises AZD6738.

In some embodiments, the treatment is a treatment that includes an ATR inhibitor and optionally one or more other agents, such as one or more chemotherapeutics.

In some embodiments, the treatment comprises one or more standard of care treatments. For example, the treatment can comprise one or more of gemcitabine, olaparib, paclitaxel, carboplatin, acalabrutinib, durvalumab and cisplastin. The treatment can for example comprise AZD6738 and gemcitabine and/or cisplastin.

AZD6738 is being tested in several clinical trials including for example NCT03022409, NCT03770429 and NCT01955668. The dosing of AZD6738 can be 20-160 mg twice daily and optionally continuous dosing for a minimum of 9 days and a maximum of 28 days. Alternatively, AZD6738 can be administered on a 9-28 day cycle and can be administered as an oral formulation.

A further aspect the ATR inhibitor is selected from AZD6738 and analogs thereof. In one embodiment, the ATR inhibitor is AZD6738. In an embodiment, the analog of AZD6738 is AZ20.

The ATR inhibitor can for example be formulated for oral delivery.

In one embodiment, the treatment comprises an ATR inhibitor and an CDK11 inhibitor.

In one embodiment, the combination is or comprises OTS964 and AZD6738.

The biological sample can be any sample from the patient comprising cancer cell molecules that can be used for assessing mutational status of FBXW7 or upregulation of CCNL1. For example, the biological sample can comprise cancer cell nucleic acids, optionally tumor genomic DNA or cancer cell transcripts, and can include a tumor sample, for example obtained by biopsy of the tumor or a liquid biopsy comprising circulating tumor cells or circulating tumor DNA. The liquid biopsy can be a blood sample, or a fraction thereof, for example circulating tumor cell fraction, or nucleic acid fraction. The samples can be subjected to one or more steps to isolate nucleic acids. The biological can also comprise a cellular protein extract, for example in methods that test for CCNL1 protein levels. In such case a tumor sample or in the case of blood cancers, a blood sample, can be taken and a cancer cellular protein fraction isolated.

Accordingly, in some embodiments, the biological sample comprises cancer cell nucleic acids. In other embodiments the biological sample comprises cancer cell protein fraction.

In some embodiments, the testing comprises assaying for one or more deleterious mutations in the FBXW7, optionally in the FBXW7 substrate-binding domain. For example, the testing can comprise sequencing a FBXW7 transcript or part thereof for example using a method involving RT-PCR of the cancer cell mRNA and sequencing. The part can for example be the exons containing the substrate binding domain region.

In another embodiment, the testing further comprises comparing the FBXW7 transcript sequence to wild type FBXW7 to identify the presence or absence of a deleterious mutation.

In an embodiment, the deleterious mutation assayed is any of a point mutation, truncation or deletion or combinations thereof. For example, in some embodiments selected combinations of point mutations are assayed. In an embodiment, the deleterious mutation(s) assayed is one or more of the mutations listed in Table 1. Any subcombination of the mutations listed in Table 1 is contemplated, for example, any 2, 3, 4, 5, 6, 7, or 8 or more mutations listed in Table 1. For example, the mutation can encode a R505 mutation, optionally R505C or R505L. For example, if a patient has a particular cancer, one or more mutations associated with the particular cancer may be assayed.

The deleterious mutation of FBXW7 can as shown herein result in an increase in the cellular levels of CCNL1 proteins or mRNA relative to cellular levels of CCNL1 in a cell without the alteration, an increase in activity of CDK11 relative to activity of CDK11 in a cell without the alteration, hypersensitivity to inhibition by the CDK11 inhibitor, an increase in activity of ATR relative to activity of ATR in a cell without the alteration, and/or hypersensitivity to inhibition by the ATR inhibitor. As demonstrated herein, CCNL1 is upregulated upon loss of function of FBXW7. CCNL1 is upregulated in a variety of cancers, in particular by gene amplification or gain.

Accordingly, in a further aspect, the testing comprises measuring cellular levels of CCNL1 protein or mRNA. In an embodiment, the cellular levels of the CCNL1 mRNA are measured by RT-PCR method. In a further embodiment, the cellular levels of CCNL1 is measured using a standard polypeptide assay, or by immunohistochemistry of a tumor sample or by immunocytochemistry of a cell sample.

CCNL1 upregulation can be assayed by assessing for amplification or gain of CCNL1. In a further aspect, the testing comprises determining if CCNL1 is amplified or gained. In some embodiments, amplification and/or gain is assessed using qPCR, RNAseq and/or fluorescence in situ hybridization (FISH).

Sequencing of the regions of interest is used for example to detect the FBXW7 mutation. Primers can be designed for example within 200 nucleotides upstream and 200 nucleotides downstream of the mutation resulting in the R505 mutation. CCNL1 copy number can for example be assessed using the method shown in Muller et al 2006, incorporated herein by reference. Primers can be designed to produce a desired transcript length for sequencing for example a transcript with a nucleotide length of including or between 100 to 600 nucleotides in length.

In another embodiment, CCNL1 upregulation is assayed by testing for a mutant CCNL1. For example, mutations in CCNL1 have been reported on cBioportal and TCAG including a frameshift at codon 163 which is positioned proximal to the phosphodegron domain which is required for FBXW7 binding.

Upregulated CCNL1 can be assessed by testing for increased levels of CCNL1 protein and/or transcript in cancer cells. In some embodiments, the biological sample taken from the patient is used to identify if the patient has an increased level of CCNL1 protein and/or transcript levels. Such patient may optionally be further assessed to determine if the patient has a deleterious alteration in FBXW7 such as a deleterious mutation and/or a CCNL1 amplification or gain. Tumors exhibiting high CCNL1 levels may contain a FBXW7 deleterious mutation or amplification/gain of CCNL1.

The methods for testing for a FBXW7 mutation or CCNL1 level can comprise one or more steps as described in the Examples. For example, the level of CCNL1 can be detected by an antibody based method, optionally using the antibody described in the Examples.

In another aspect, the method further comprises treating the patient with an effective amount of a CDK11 inhibitor and/or ATR inhibitor treatment as described herein.

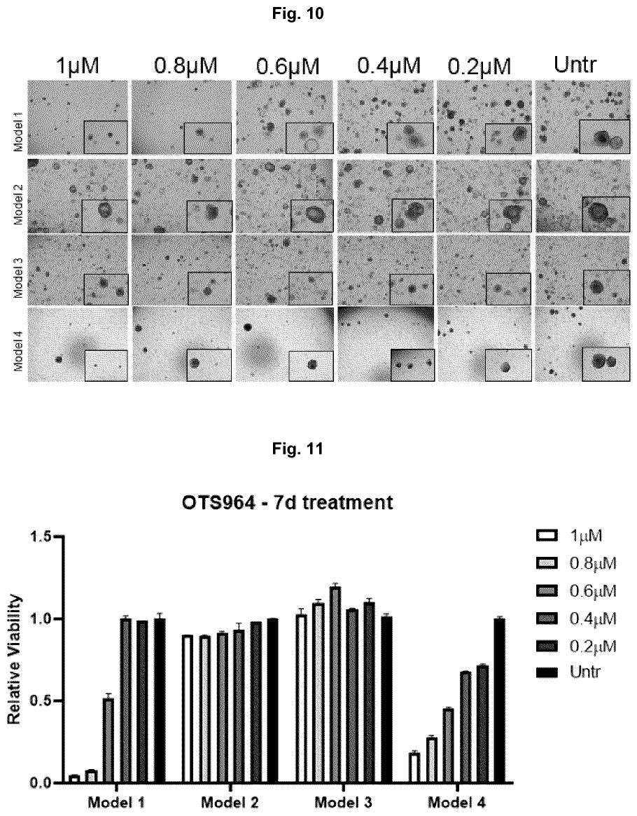

As shown herein, the therapeutic window in cancer cells harboring a FBWX7 mutation compared to wild-type is as demonstrated in FIG. 9, to be at least 5 fold. This may be an underestimation since inhibition in wildtype cells was only seen at the highest dose tested (1 uM).

Also provided are uses, for example use of a CDK11 inhibitor and/or ATR inhibitor treatment for treating a patient afflicted with a cancer having i) a deleterious mutation in F box WD-repeat containing protein (FBWX7), optionally in its substrate binding domain or ii) upregulation of CCNL1, for example gain or amplification of CCNL1.

A further aspect includes a kit comprising one or more reagents for performing an assay as described herein. In an embodiment, the kit is used for selecting and treating a patient afflicted with a cancer likely to benefit from a CDK11 inhibitor and/or ATR inhibitor treatment.

Yet a further aspect includes a package comprising a vial comprising a CDK11 inhibitor and/or ATR inhibitor and a label or instructions, for administering to a patient with a FBWX7 deleterious mutation or upregulation of CCNL1. In an embodiment, the package is for use in a method described herein.

The methods, uses, kits and packages can comprise pharmaceutical compositions comprising one or more CDK11 inhibitors and/or ATR inhibitors.

Pharmaceutical compositions described herein can be prepared by any method known in the art of pharmacology. In general, such preparatory methods include the steps of bringing a compound described herein (the “active ingredient”) into association with a carrier and/or one or more other accessory ingredients, and then, if necessary and/or desirable, shaping and/or packaging the product into a desired single- or multi-dose unit.

Injectable preparations, for example, sterile injectable aqueous or oil based suspensions can be formulated according to the known art using suitable dispersing or wetting agents and suspending agents. The sterile injectable preparation can be a sterile injectable solution, suspension or emulsion in a nontoxic parenterally acceptable diluent or solvent. Among the acceptable vehicles and solvents that can be employed are water, Ringer's solution, U.S.P. and isotonic sodium chloride solution. In addition, sterile, fixed oils are conventionally employed as a solvent or suspending medium.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as sodium carbonate, e) wetting agents such as, for example, cetyl alcohol and glycerol monostearate, f) absorbents such as kaolin and bentonite clay, and g) lubricants, and mixtures thereof. In the case of capsules, tablets and pills, the dosage form may comprise buffering agents.

The above disclosure generally describes the present application. A more complete understanding can be obtained by reference to the following specific examples. These examples are described solely for the purpose of illustration and are not intended to limit the scope of the application. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present disclosure:

### EXAMPLES

### Example 1: Mutational Events in FBXW7 and CCNL1 in Cancers

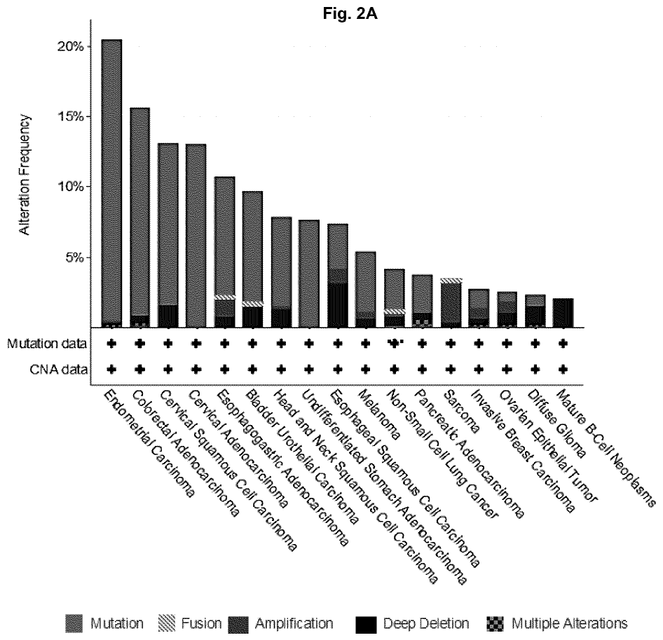

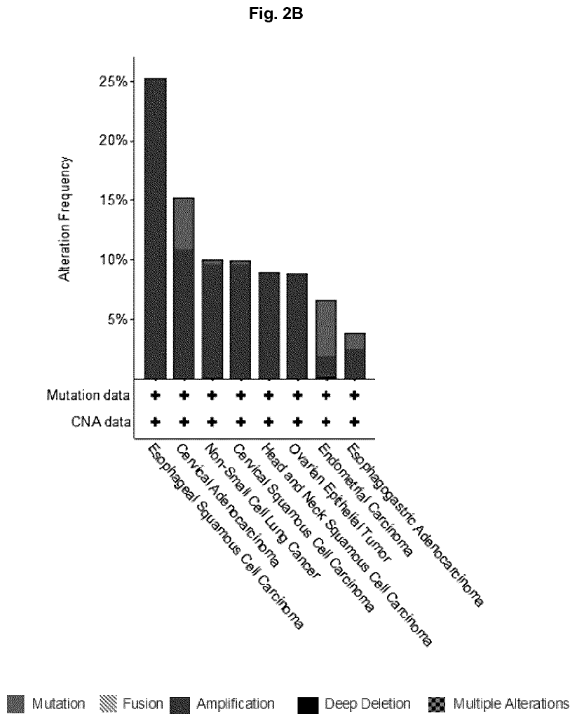

Mutations in FBXW7 are common across cancers, and regions of mutational hotspots are located within substrate-recognition domains (WD-repeat domains) of the protein (FIG. 1). Therefore, these may interfere with substrate recognition, the regulation of their intracellular levels by the ubiquitin system, and therefore, their accumulation leading to hyperactivity contributing to tumorigenesis. FBXW7 mutations are highly prevalent, and occur in a wide range of cancers, most commonly uterine carcinosarcoma, squamous lung cancer, esophageal cancer, head and neck cancer and cervical cancer (FIG. 2A and Table 2). Amplification of CCNL1 occurs in a similar subset of cancers containing FBXW7 mutations (FIG. 2B and Table 3), and these two mutational events appear to frequently be mutually exclusive (FIG. 3) raising a possibility that these two genes may be acting in the same pathway and when mutated lead to tumorigenesis. FBXW7 mutations and amplification of CCNL1 occur most frequently in cancers of squamous origin (FIG. 2B).

Using genome-wide CRISPR-Cas9 essentiality screens it is shown that in a novel model of FBXW7-dependent cell proliferation, CCNL1 is synthetic lethal to FBXW7 mutations.

TABLE 1
Possible FBXW7 Mutations in Cancer Cells
FBXW7 mutations

| | | | | |
|---|---|---|---|---|
| R505C | E287K | K167T | R133Ifs*30 | T536R |
| R465H | E287V | K185N | R14_T15delinsQTP | T576M |
| R505H | E369* | K239Q | R14* | T653A |
| R658Q | E471Kfs*27 | K299Sfs*2 | R14Q | T653M |
| R465C | E588K | K371* | R179H | V143D |
| R479G | E655* | K374Q | R20I | V341Sfs*22 |
| R505G | E693K | K404E | R222* | V37Rfs*24 |
| R479P | E694* | L157Yfs*15 | R224* | V405Ffs*10 |
| R479Q | E97* | L251F | R224Q | V418M |
| R505L | E99* | L290F | R278* | V418Qfs*9 |
| R465P | F161L | L301P | R278Q | V475del |
| Y545C | F241L | L320I | R309Afs*33 | V485Ffs*13 |
| A252Lfs*2 | F280L | L443F | R309H | V523L |
| A305Sfs*13 | FBXW7- | L457* | R339* | V525E |
| | ARHGAP10 | | | |
| A481V | FBXW7-MGST2 | L483I | R357_I361del | V554L |
| A502V | FBXW7-SETD7 | L494* | R357K | V59F |
| A626D | FBXW7-SH3D19 | L497F | R35C | V672M |
| A626T | FBXW7- | L547Ffs*4 | R367* | V8L |
| | TMEM126B | | | |
| C308* | G233C | L577S | R393* | V8M |
| C466* | G368E | L651* | R393Efs*2 | W237* |
| C506Y | G378* | L660Qfs*47 | R393Q | W321* |
| C533R | G391D | L682Afs*12 | R441Q | W321* |
| D109Rfs*2 | G411S | LRBA-FBXW7 | R441W | W365* |
| D126Ifs*43 | G423* | M240Cfs*14 | R473Efs*25 | W365S |
| D129A | G423A | M268Dfs*18 | R473Kfs*4 | W526R |
| D135Y | G423V | M33I | R479* | W649* |
| D279Y | G437* | N216Mfs*23 | R479G | W673* |
| D300Y | G437R | N542I | R479L | R689W |
| D380_H382dup | G499D | N621Kfs*23 | R479P | R83I |
| D399H | G517E | N679S | R479Q | S191* |
| D399V | G517R | P247T | R513W | S25* |
| D480Y | G571V | P274Vfs*11 | R543G | S282* |
| D520E | G571W | P298L | R543K | S294* |
| D520H | G75* | P298T | R543Kfs*2 | S396N |
| D520N | G93* | P373S | R658* | S398F |
| D560H | G9S | P620R | Y291Cfs*25 | S398Y |
| D560V | H382N | PPP4R2-FBXW7 | Y519* | S426L |
| D600H | H420Qfs*13 | Q120* | Y545* | S436Lfs*63 |
| D600N | H470Y | Q127* | Y545C | S438F |
| D600Y | H500D | Q277E | S668Vfs*39 | S476I |
| D642G | H500Q | Q306* | S678* | S476R |
| E102K | H500R | Q358* | S678* | S516R |
| E113* | H500Y | Q388Tfs*5 | S86L | S546* |
| E177K | H52Y | Q44* | S86L | S546* |
| E177Q | H540N | Q492Rfs*6 | SLC20A2-FBXW7 | S546L |
| E192A | H575P | Q508* | T196S | S558F |
| E248* | H580Y | Q581Pfs*24 | T263Nfs*24 | S582L |
| E248D | I257Nfs*11 | Q618* | T385I | S585T |
| E273* | I330Dfs*2 | Q73* | T385K | S601F |
| K167T | I384Wfs*2 | Q95* | T482Ffs*16 | S625G |
| R13* | S641* | S668Efs*26 | T536P | S640Tfs*7 |
| R513W | R543G | R543K | R543Kfs*2 | R658* |

*premature stop codon; position references are with respect to Accession # Q969H0

TABLE 2
Cancer Types harboring FBXW7 mutations
Endometrial Carcinoma
Colorectal Adenocarcinoma
Cervical Squamous Cell Carcinoma
Cervical Adenocarcinoma
Esophagogastric Adenocarcinoma
Bladder Urothelial Carcinoma
Head and Neck Squamous Cell Carcinoma
Undifferentiated Stomach Adenocarcinoma
Esophageal Squamous Cell Carcinoma
Melanoma
Non-Small Cell Lung Cancer
Pancreatic Adenocarcinoma
Sarcomal
Invasive Breast Carcinoma
Ovarian Epithelial Tumor
Diffuse Glioma
Mature B-Cell Neoplasms
Pheochromocytoma
Hepatocellular Carcinoma
Ocular Melanoma
Pleural Mesothelioma
Glioblastoma
Prostate Adenocarcinoma
Well-Differentiated Thyroid Cancer
Renal Clear Cell Carcinoma
Renal Non-Clear Cell Carcinoma
Adrenocortical Carcinoma
Cholangiocarcinoma
Leukemia Miscellaneous Neuroepithelial Tumor
Seminoma
Non-Seminomatous Germ Cell Tumor
Thymic Epithelial Tumor

TABLE 3
Cancer Types harboring CCNL1 amplification
Esophageal Squamous Cell Carcinoma
Cervical Adenocarcinoma
Non-Small Cell Lung Cancer
Cervical Squamous Cell Carcinoma
Head and Neck Squamous Cell Carcinoma
Ovarian Epithelial Tumor
Endometrial Carcinoma
Esophagogastric Adenocarcinoma
Bladder Urothelial Carcinoma
Pancreatic Adenocarcinoma
Colorectal Adenocarcinoma
Mature B-Cell Neoplasms
Melanoma
Thymic Epithelial Tumor
Seminoma Invasive Breast Carcinoma
Prostate Adenocarcinoma
Pleural Mesothelioma
Adrenocortical Carcinoma
Sarcoma
Renal Clear Cell Carcinoma
Renal Non-Clear Cell Carcinoma
Hepatocellular Carcinoma
Glioblastoma
Diffuse Glioma
Cholangiocarcinoma
Leukemia
Pheochromocytoma
Miscellaneous Neuroepithelial Tumor
Undifferentiated Stomach Adenocarcinoma
Non-Seminomatous Germ Cell Tumor
Well-Differentiated Thyroid Cancer
Ocular Melanoma

### Example 2: Genome Wide CRISPR-Cas9 Screen Reveals Genetic Determinants of PORCN Inhibitor Sensitivity

HPAF-II cells harbor a mutation in RNF43 a negative regulator of the Wnt signaling pathway and as a result these cells are exquisitely dependent on the presence of Wnt ligands. Treatment of these cells with the Porcupine inhibitor (enzyme required for the secretion and activity of Wnt growth factors) LGK974 leads to cell cycle arrest and strong inhibition of proliferation. Genome wide CRISPR-Cas9 screen was used to identify genes underlying PORCN inhibitor sensitivity, specifically, gene knockouts mediating LGK974 resistance. To do this, the RNF43 mutant cell line HPAF-II was transduced with the genome-wide TKO gRNA library and treated with a LD90 dose of the PORCN inhibitor LGK974 (Liu et al 2013). Under these conditions, the vast majority of cells undergo cell cycle arrest, but a few clones bypassed this process and emerged during the selection. Next generation sequencing of the resistant clones identified gRNA targeting several known negative regulators of Wnt signalling—including APC and AXIN1 that re-activated the ßcatenin pathway distal to the receptor complex. Another gene that was identified was the E3 ubiquitin ligase substrate specific receptor FBXW7, a tumour supressor gene, which has previously been linked to regulation of MYC and CyclinE levels.

As validation, each of the genes discovered in this LGK974 positive selection screen were knocked-out in HPAF-II cells and the sensitivity to LGK974 was determined using clonogenic assays. The assay tested HPAF-II wild type cells, HPAF-II wild type cells treated with LGK974, HPAF-II cells with the gene APC knocked out and treated with LGK974, HPAF-II cells expressing a degradation resistant mutant (DegR)-ßCatenin and treated with LGK974, HPAF-II cells with the AXIN gene knocked out and treated with LGK974, and HPAF-II cells with the FBXW7 gene knocked out and treated with LGK974. Whereas LGK974 treatment robustly arrested the growth of HPAF-II wildtype cells (containing an RNF43 mutation), knockout of APC and AXIN1 fully rescued the growth of these cells in the presence of the inhibitor. The induced resistance to PORCN inhibitor was not as robust for knockout of FBXW7 but nevertheless led to partial resistance to the inhibitor.

### Example 3: Identification of Context-Specific Fitness Genes

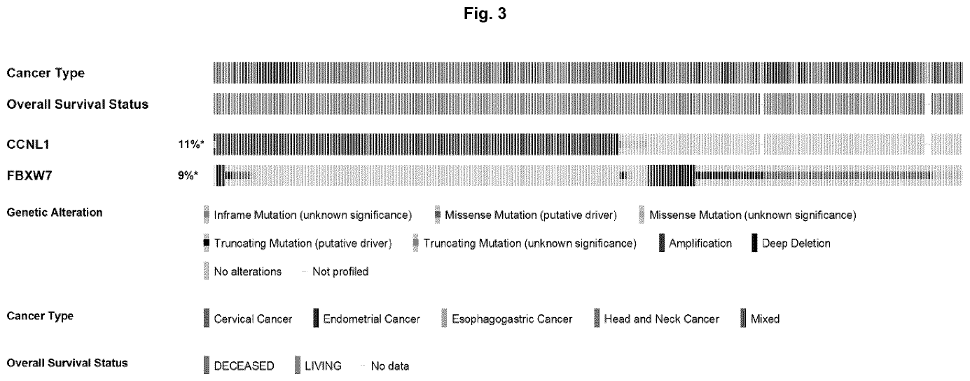

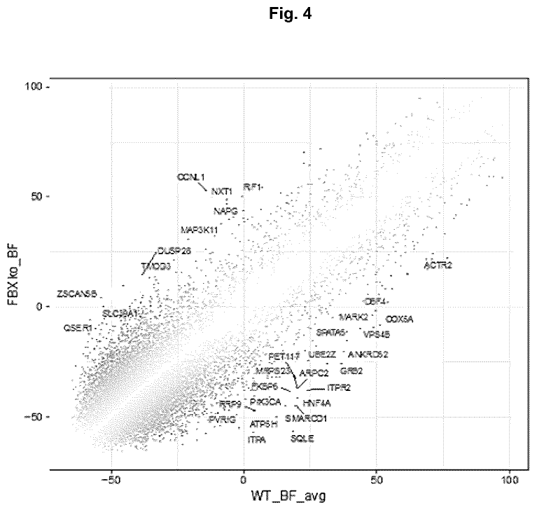

Using the methods of Example 8, a dropout genome-wide CRISPR essentiality screen was performed in these cells and compared to the otherwise isogenic wild-type HPAF-II cells, illustrating the genetic network underlying the growth properties of FBXW7^{-/-} HPAF-II cells. Results of the screens were analyzed using BAGEL (Hart and Moffat, 2016) that ascribes a degree of essentiality to support the cellular growth for each gene. Some genes were identified to be essential only in the FBXW7^{-/-} context, and are therefore referred to as context-specific fitness genes, including the poorly characterized gene CCNL1 that encodes for the cyclin protein CCNL1 (FIG. 4). This synthetic lethal genetic interaction was further validated using proliferation assays (FIG. 5A) and multicolor-competition assays (FIG. 5B) together demonstrating the context-dependent essentiality of CCNL1 in FBXW7^{-/-} cells with little effect of CCNL1 knockout on wildtype cells.

### Example 4: CCNL1 as a Novel Substrate of FBXW7

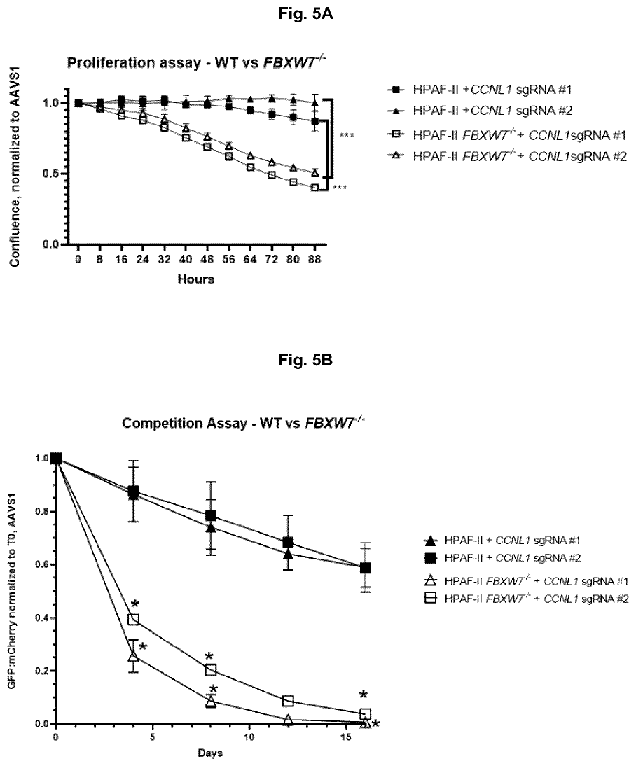

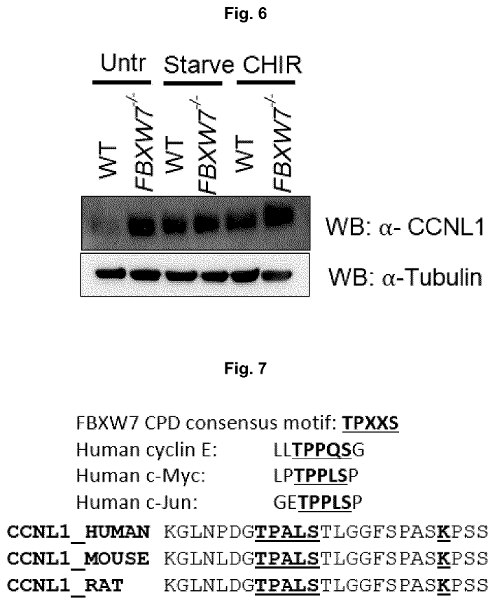

Using the methods of Example 8, the functional role of FBXW7 is a substrate recognition component of an E3 ligase complex, tasked with regulating protein abundance of substrates. Wildtype HPAF-II cells express very little CCNL1 in exponentially growing cells, while FBXW7^{-/-} HPAF-II cells show a robust increase in CCNL1 expression (FIG. 6). CCNL1 does indeed contain a potential FBXW7 phosphodegron recognition motif (required for FBXW7 binding), highly conserved across species (FIG. 7).

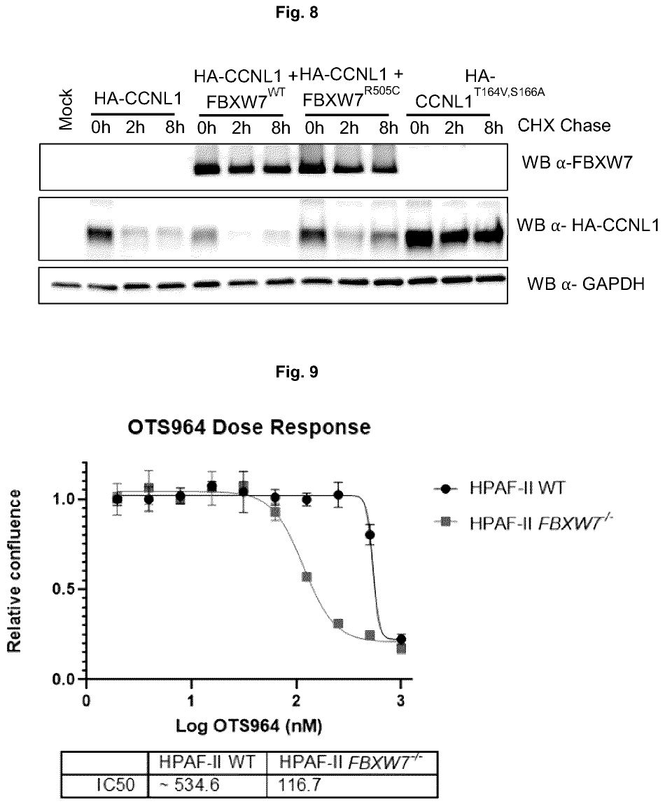

Using the methods of Example 8, a co-expression analysis followed by cycloheximide chase illustrated the effect of FBXW7 expression on CCNL1 stability. When CCNL1 and FBXW7^{WT} are overexpressed together, a decrease in total CCNL1 protein levels and a reduction of its half-life was observed, while expression of CCNL1 alone or co-expression with the FBXW7R^{505C} mutant that is found in cancer showed higher overall CCNL1 levels and increased half-life (FIG. 8). Mutation in the substrate-recognition domain of FBXW7 lead to a robust stabilization of CCNL1 levels. CCNL1 expression could be induced in wild-type HPAF-II cells following serum starvation, suggesting that CCNL1 accumulates at the exit of mitosis or during G0 (FIG. 6). Furthermore, given that phosphorylation of substrates are usually required for binding to F-Box proteins, we treated cells with the GSK3 inhibitor CHIR99021 and observed increased levels of CCNL1 (FIG. 6). This demonstrates that CCNL1 is a novel substrate of the FBXW7 SCF E3 ubiquitin ligase.

### Example 5: Sensitivity to CDK11 Inhibition

Using the methods of Example 8, immunoprecipitation paired with mass spectrometry was performed to identify potential interactors that could be involved in cell cycle progression, and CDK11B was identified. Using the methods of Example 8, it was tested whether FBXW7 mutant cells, which were shown in Example 4 to have higher CCNL1 and would therefore suggest higher CDK11 activity, would be hypersensitive to CDK11 inhibition. Proliferation of FBXW7^{-/-} HPAF-II cells displayed a striking increase in sensitivity to the CDK11 inhibitor (OTS964), when compared to the wildtype HPAF-II cells, confirming the role of the CCNL1-CDK11 axis in FBXW7^{-/-} mutant cancers (FIG. 9). FBXW7 mutant cells are at least 5 times more sensitive to CDK11 inhibition using the CDK11 inhibitor OTS964 (FIG. 9).

### Example 6: FBXW7 Mutations in Organoids and Cell Models Sensitive to OTS964 Treatment

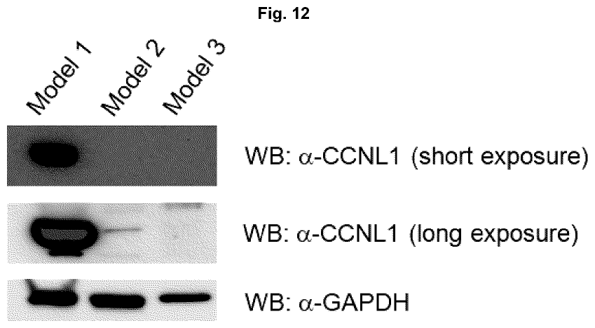

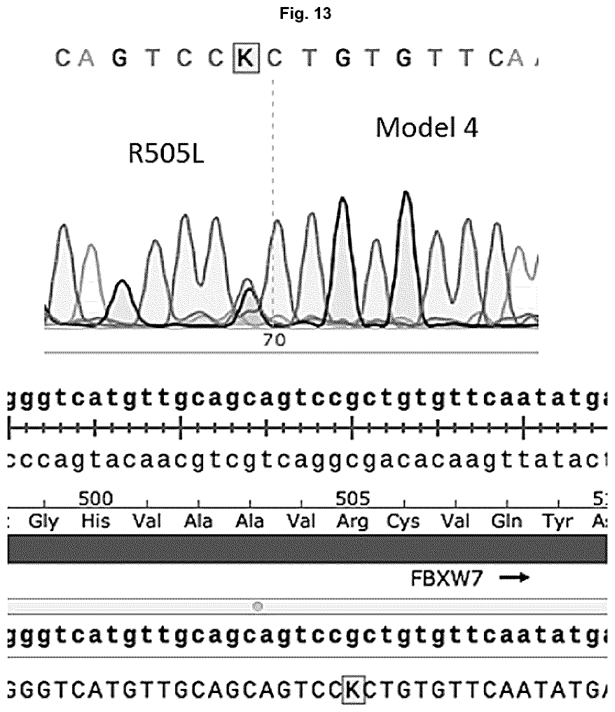

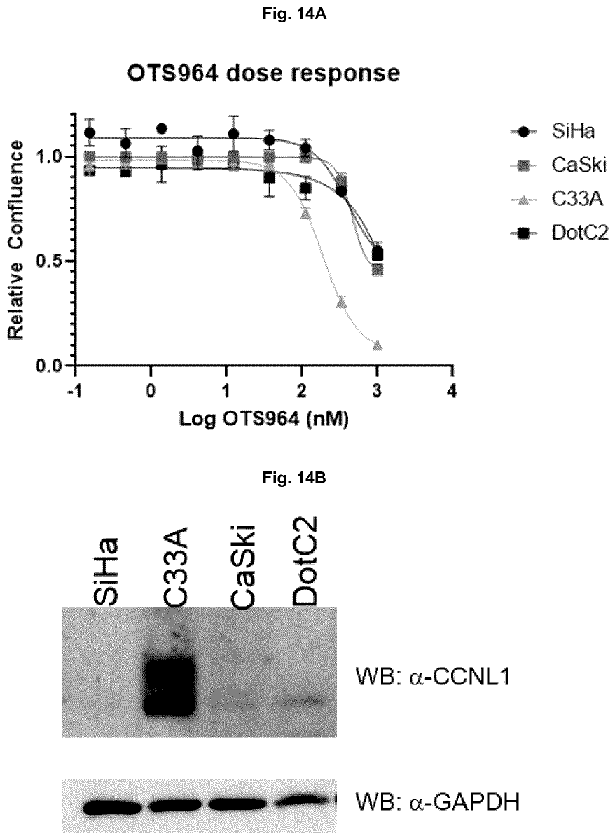

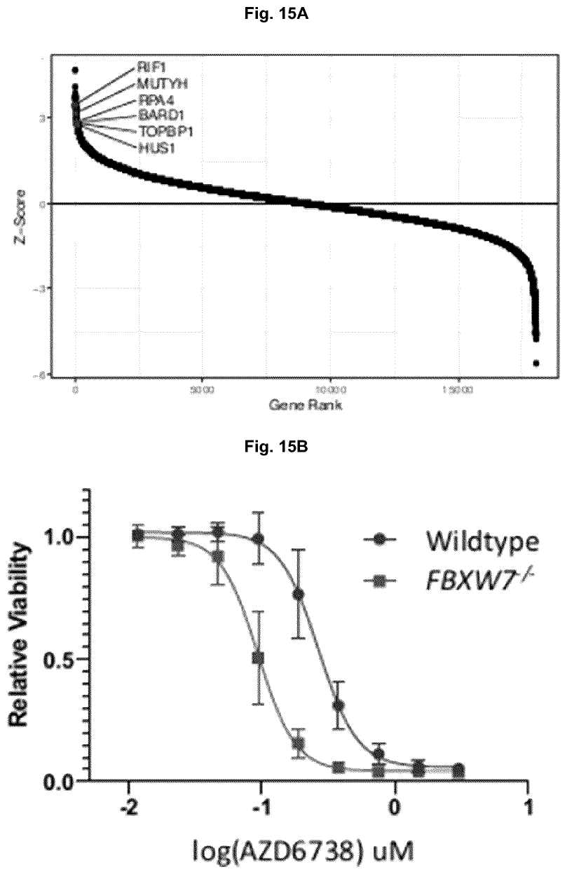

Cervical cancer patient-derived cancer organoids were treated with dose-response of OTS964 for 7 days. Following treatment, organoids were imaged, and viability was measured using Cell Titer Glo. Viability was normalized to untreated conditions (FIGS. 10 and 11). This illustrates that a subset of organoid models is sensitive to OTS964. Western-blotting, using the methods of Example 8, of organoid lysates indicates high levels of CCNL1 expression in OTS964-sensitive model 1 (FIG. 12). This indicates that Cervical cancer organoids that are sensitive to OTS964 show high levels of CCNL1 expression. Sanger sequencing results revealed FBXW7 mutations in the two OTS964 sensitive cervical cancer organoid models (FIG. 13). R505L is a well-characterized FBXW7 hot-spot mutation (FIG. 13). This demonstrates that OTS964 responders have mutations in FBXW7.

### Example 7: FBXW7^{-/-} Cells are Sensitive to Inhibition of ATR

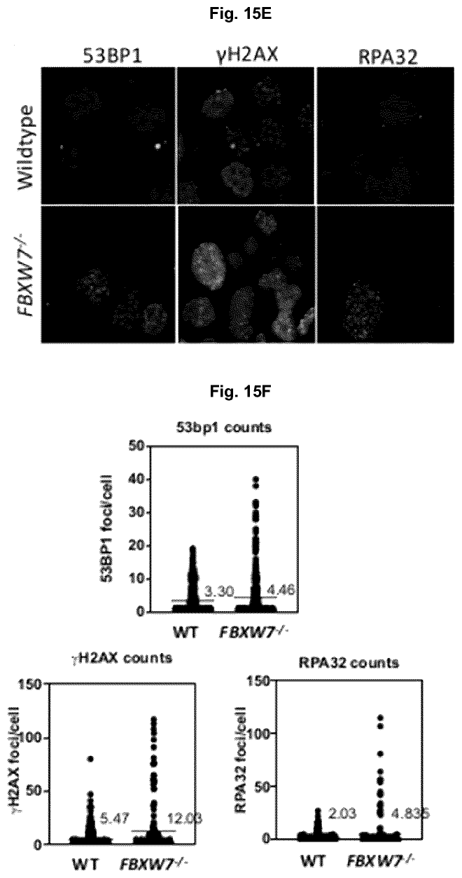

Using the methods of Example 8, genetic vulnerabilities identified in the FBXW7^{-/-} cells were highlighted, and numerous genes comprising a signature for DNA replication stress were identified as essential only in the FBXW7^{-/-} cell line (FIG. 15A). Using the methods of Example 8, HPAF-II wildtype and FBXW7^{-/-} were treated with an inhibitor of ATR (Ataxia telangiectasia and Rad3 related) AZD6738, with FBXW7^{-/-} cells being much more susceptible to inhibition of ATR (FIG. 15B). Further, whole cell lysates from both HPAF-II wildtype and FBXW7^{-/-} cells were immunoblotted for phosphorylated CHK1, a marker of DNA replication stress and ATR activity, suggesting that highly active ATR in FBXW7^{-/-} may be responding to higher levels of DNA damage (FIG. 15C). In addition, the cervical cell line panel was assessed for response to AZD6738, and the FBXW7 mutated cell line C33A demonstrates the highest susceptibility to this inhibitor, with 4-6 fold enhanced activity compared to Caski and SiHa cell lines, which are wild-type for FBXW7 (FIG. 15D). Increased DNA replication stress in FBXW7 mutant cells is predicted to be associated to increase DNA damage levels. To assess the level of DNA damage response (DDR), both wildtype and FBXW7^{-/-} cells were subjected to immunofluorescence staining to detect several nuclear markers of DNA damage responses—53BP1, γH2AX, and RPA32. In all cases, these markers were highly upregulated in FBXW7^{-/-} cells when compared to wild type cells. We conclude that FBXW7−/− cells have increased DNA damage responses (DDR) as a result of increased DNA replication stress. This enhanced level of DDR is required for FBXW7^{-/-} to survive, providing a vulnerability and a tractable approach to targeting FBXW7 mutated tumours in the clinic with ATR inhibitors.

### Example 8: Material and Methods

Cell Culture and Treatments

HPAF-II, C33A, SiHa HEK293T cells (ATCC) were grown in DMEM (4.6 g/L D-glucose, L-glutamine)+10% fetal bovine serum+5% Anti-Anti at 37° C. and 5% CO2. Caski cells were grown in RPMI-1640+10% fetal bovine serum+5% Anti-Anti at 37° C. and 5% CO2. For serum starvation, regular growth media was removed, and replaced with DMEM (4.6 g/L D-glucose, L-glutamine)+5% Anti-Anti for 18 h. CHIR99021 was used at 4 nM.

Establishment of Knockout Cell Lines

HPAF-II mutant cells (APC-truncation, AXIN-knockout, FBXW7-knockout) were transfected via electroporation using the Neon system (Thermo) under the following conditions; 2 μg of DNA (sgRNA construct pX330) 1150V, 30 ms and 2 pulses. Cells were allowed to recover for 2 days before the addition of 100 nM LGK-974 to enrich for mutations driving Wnt pathway activation. Cell lines were validated for editing using TIDE (tracking of insertions and deletions (Brinkman et al 2014).

Lentiviral Production

HEK293T cells were transfected with 6.5 μg lentiviral plasmid, 5.9 μg packaging plasmid (pSPAX) and 0.65 μg envelope plasmid (pMD.26), using polyethylenimine transfection. 24 h post transfection, media was refreshed, and cells incubated for 24 h. Lentivirus was harvested, aliquoted, and stored at −80° C. until use.

Genome-Wide Screens

Positive Selection

HPAF-II cells were infected with the Toronto knockout library version 1 (TKOv1)—a pooled sgRNA lentiviral library (Hart et al2015) at a multiplicity of infection of 0.3, in the presence of 8 μg/ml polybrene (Sigma) for 24 h. Cells were treated with 2 μg/ml puromycin for 48 h. 7d post-selection, cells were split into treatment groups—one using an LD90 dose of LGK974 at 20 nM, and the second a DMSO control; duplicates were included for both treatment arms. LGK974 treatment was harvested at day 28, and DMSO treatment at day 31. Genomic DNA extracted using the QIAmp DNA Blood Maxi Kit (Qiagen). gDNA samples were amplified, and barcoded using i5 and i7 adaptor primers for Illumina next generation sequencing. Barcoded PCRs were sequenced with the Illumina HiSeq2500. Sequenced gRNAs were mapped to the TKOv1 library using MaGECK 0.5.3, and read counts were normalized by total reads per sample before averaging biological replicates and determining gRNA enrichment.

Dropout

HPAF-II WT and FBXW7 were infected with the Toronto knockout library version 3 (TKOv3)—a pooled sgRNA lentiviral library (Hart et al2017) at a multiplicity of infection of 0.3, in the presence of 8 μg/ml polybrene (Sigma) for 24 h. Cells were treated with 2 μg/ml puromycin for 48 hours. Following selection, pooled cells were split into three equal replicates, and passaged every 4 days for 24 days, maintaining 18 million cells/replicate. Cell pellets at T=0, 12 and 24 days were collected, and genomic DNA extracted using the QIAmp DNA Blood Maxi Kit (Qiagen). gDNA samples were amplified, and barcoded using i5 and i7 adaptor primers for Illumina next generation sequencing. Barcoded PCRs were sequenced with the Illumina HiSeq2500 with read depths of 200-fold coverage. Sequenced gRNAs were mapped to the TKOv3 library using MaGECK 0.5.3 (Li et al 2014). Read counts were normalized and fold-change of gRNA distribution compared to T=0 was calculated using the BAGEL package (Hart et al 2016). BAGEL analysis was performed, and Bayes Factors were compared between HPAF-II wildtype and FBXW7^{-/-} cells.

Proliferation Assay

HPAF-II wildtype and FBXW7−/− cells expressing Cas9 were infected with pLentiguide-2A-GFP virus stocks at an MOI of ˜0.3 in the presence of 8 μg/ml polybrene. Cells were infected overnight, and treated with 2 μg/ml puromycin for 48 hrs. Following selected, fresh media was added, and cells were left for 24 hrs to grow. Cells were seeded to ˜2500 cells/well in triplicate in a 96-well plate, and left overnight to attach. Plates were moved to the Incucyte (Sartorius) and confluence was tracked over time. Cell confluence in each line was normalized to AAVS1-infected cells.

Multicolour Competition Assay

HPAF-II wildtype and FBXW7−/− cells expressing Cas9 were infected with pLentiguide-2A-GFP or pLentiGuide-2A-mCherry-AAVS1 virus stocks at an MOI of ˜0.3 in the presence of 8 μg/ml polybrene. Cells were infected overnight, and treated with 2 μg/ml puromycin for 48 hrs. Following selected, fresh media was added, and cells were left for 24 hrs to grow. Each gRNA was mixed 1:1 with pLentiGuide-2A-mCherry-AAVS1 expressing cells, and GFP:mCherry ratios were measured by flow cytometry (Beckman Coulter CytoFLEX) every 4 days for 16 days. Data were normalized to AAVS1 controls.

Dose-Response Assays

All cells were seeded in triplicate at 2500 cells/well. The following day, a dose-response treatment of OTS964 (Cayman Chemicals) or AZD6738 (SelleckChem). At 3d and 7d of treatment, respectively cell viability was determined using CellTiter-Glo (Promega) as per manufacturer's instructions.

Western Blotting

All samples were lysed in 4× Laemmli Sample Buffer (50 mM Tris-HCl pH 6.8, 2% SDS, 10% glycerol, 1% β-mercaptoethanol, 12.5 mM EDTA, 0.02% bromophenol blue). Lysates were sonicated, boiled, and centrifuged to pellet insoluble material. Approx 10 μg were loaded per sample on a 4-15% SDS-PAGE Stain-Free TGX BioRad precast gel. Gels were run at 150V for approximately 60 minutes. Gels were transferred to methanol-activated PVDF at 90V for 120 minutes. Membranes were blocked in 5% milk in Tris-buffered Saline (pH 7.4)+1% Tween-20 (TBS-T) for 1 hr, and incubated with corresponding primary antibodies overnight. The following day, membranes were washed 4× in TBS-T, and incubated with corresponding secondary antibodies for 1 hour, in 5% milk in TBS-T, at room temperature with agitation. Membranes were washed, and imaged using SuperSignal West Pico PLUS chemiluminescent substrate (Thermo Fisher) and imaged on the BioRad Chemidoc-MP.

Immunoprecipitation-Mass Spectrometry

15-cm plates of 80% confluent HPAF-II cells were washed 2× in ice-cold PBS and lysed in 1 mL TAP lysis buffer (0.1% NP-40, 10% glycerol, 50 mM HEPES pH 8, 150 mM NaCl, 2 mM EDTA, 2 mM DTT, 10 mM NaF, 250 uM NaOVO3, 50 mM β-glycerolphosphate), with end-over-end rotation at 4° C. for 30 minutes. Lysate was centrifuged at 12,000×g at 4° C. for 20 minutes to pellet insoluble material. 400 uL of lysate was incubated with 10 μg Rb-anti CCNL1 antibody (Bethyl) or human IgG, overnight at 4° C. with end-over-end rotation. The following day, 30 uL Protein G-conjugated agarose beads (Roche) were equilibrated with TAP buffer, and added to lysates. Lysate and bead mixture was incubated at 4° C. for 3 hours with end-over-end rotation. Following incubation, beads were washed extensively first in TAP buffer, and then in 50 mM ammonium bicarbonate. 2 uL of sequencing-grade trypsin (Promega) was added to slurry, and incubated overnight at 37° C. The following day, supernatant was removed and dried using a SpeedVac. Protein was desalted using Pierce C18 Spin tips (Thermo Fisher), vacuum dried, and resuspended in Buffer A (1% formic acid). Samples were run on QExactive HF mass spectrometer, peptides searched in ProHits database.

Cycloheximide Chase

HEK293T cells were transfected with 2 μg total DNA using polyethylenimine (PEI) for 18 hrs. Following transfection, cells were split into corresponding conditions. The following day, cells were treated with 10 μg/ml cycloheximide, and harvested at corresponding timepoints. Constructs are as follows: pCDNA3.1-HA-CCNL1, pCDNA5-BirA-FBXW7, pCDNA5-BirA-FBXW7^{R505G}.

Sanger Sequencing of Patient-Derived Organoids

Organoids were harvested, and genomic DNA isolated using the PureLink genomic DNA mini kit (Invitrogen) as per manufacturers instructions. Regions of interest were PCR amplified using KAPA-HiFi polymerase (Kapa Biosytems), under standard cycling conditions. PCR amplicons were separated on a 1% agarose gel, excised, and purified using the PureLink PCR and gel purification kit (Invitrogen). 300 ng of DNA was sent for Sanger sequencing using 5 uM PCR-Fwd primer to The Centre For Applied Genomics (Toronto, ON). Sequences were aligned using SnapGene (GSL Biotech LLC).]

Immunofluorescence and Quantification

Cells were seeded on coverglass overnight. The following day, cells were fixed using 4% paraformaldehyde for 15 minutes at room temperature. For 53BP1 and γH2AX, cells were permeabilized using 0.5% Triton X-100 in 5% bovine serum albumin (BSA) in PBS for 15 minutes at room temperature, followed by a 1 hour block in 5% BSA at room temperature. Primary antibodies in 5% BSA in PBS were added overnight and stored at 4° C. The following day, coverslips were washed in PBS, incubated for 1 hr with secondary antibody for 1 hr, and mounted on slides using Vectashield+DAPI (Vector Labs). Slides were stored at 4° C. until imaging. For RPA32 staining, cells were first treated with ice-cold nuclear extraction buffer (20 mM HEPES pH 7.5, 20 mM NaCl, 5 mM MgCl₂, 1 mM DTT, 0.5% NP-40, protease inhibitor) for 10 minutes at 4° C. Cells were then fixed in 2% paraformaldehyde, and extensively washed before staining as per the above protocol. Images were collected at 63× on a Zeiss LSM750 confocal microscope. Following image collection, nuclear foci were quantified using CellProfiler.

TABLE 4
sgRNA Sequences

| sgRNA | Sequence |
|---|---|
| FBXW7 | gtggttctgaggtccgctctt (SEQ ID NO: 1) |
| BCatenin | cagaatggattccagagtcc (SEQ ID NO: 2) |
| APC | cccggcttccataagaacgg (SEQ ID NO: 3) |
| AXIN | ggagcctcagaagttcgcgg (SEQ ID NO: 4) |
| CCNL1 #1 | aagttatcaaagcagagagg (SEQ ID NO: 5) |
| CCNL1 #2 | ttgaaatcgaacaaacacat (SEQ ID NO: 6) |
| AAVS1 | gtcccctccaccccacagtg (SEQ ID NO: 7) |

TABLE 5
Antibodies

| Target | Product # | Supplier |
|---|---|---|
| Cyclin L1 | A302-059A | Bethyl Laboratories |
| FBXW7 | A301-720A | Bethyl Laboratories |
| HA-tag | 3724S | Cell Signalling Technology |
| GAPDH | MA5-15738 | Thermo Fisher |
| β-tubulin | E7C | Iowa Hybridoma Bank |
| pCHK1 S345 | 2348S | Cell Signalling Technology |
| CHK1 | 2360 | Cell Signalling Technology |
| γH2AX | 7631 | Cell Signalling Technology |
| 53BP1 | 4937 | Cell Signalling Technology |
| RPA32 | Ab2175 | Abcam |

While the present application has been described with reference to what are presently considered to be the preferred examples, it is to be understood that the application is not limited to the disclosed examples. To the contrary, the application is intended to cover various modifications and equivalent arrangements included within the spirit and scope of the appended claims.

The scope of the claims should not be limited by the preferred embodiments and examples, but should be given the broadest interpretation consistent with the description as a whole.

### CITATIONS FOR REFERENCES REFERRED TO IN THE SPECIFICATION

Ahmed, R L., et al. 2019. CDK11 loss induces cell cycle dysfunction and death of BRAF and NRAS melanoma cells. Pharmaceuticals. 12(50).
Arnedos M, Vicier C, Loi S, Lefebvre C, Michiels S, Bonnefoi H and Andre F. Precision medicine for metastatic breast cancer-limitations and solutions. Nat Rev Clin Oncol. 2015; 12(12):693-704.
Brinkman E., et al. 2014. Easy quantitative assessment of genome editing by sequence trace decomposition. Nucleic Acids Research. 42(22).
Foote, K. M. et al. Discovery of 4-{4-[(3R)-3-Methylmorpholin-4-yl]-6-[1-(methylsulfonyl)cyclopropyl]pyrimidin-2-yl}-1H-indole (AZ20): a potent and selective inhibitor of ATR protein kinase with monotherapy in vivo antitumor activity. J. Med. Chem. 56, 2125-2138 (2013).
Foote, K. M. et al Discovery and Characterization of AZD6738, a Potent Inhibitor of Ataxia Telangiectasia Mutated and Rad3 Related (ATR) Kinase with Application as an Anticancer Agent J Med Chem 2018, 61, 22, 9889-9907
Hart T., et al. 2015. High-resolution CRISPR screens reveal fitness genes and genotype-specific cancer liabilities. Cell. 163.
Hart, T., Moffat, J. 2016. BAGEL: a computational framework for identifying essential genes from pooled library screens. BMC Bioinformatics. 17(164).
Hart, T et al. 2017. Evaluation and design of genome-wide CRISPR/SpCas9 knockout screens. G3. 7(8).
Li, W., et al. 2014. MAGeCK enables robust identification of essential genes from genome-scale CRISPR/Cas9 knockout screens. Genome Biology. 15(554).
Lin, A et al. 2019. Off-target toxicity is a common mechanism of action of cancer drugs undergoing clinical trials. Science Translational Medicine. 11(509).
Liu J., et al. 2013. Targeting Wnt-driven cancer through inhibition of Porcupine by LGK974. Proceedings of the National Academy of Sciences. 110(50).
Loyer, P., et al. 2008. Characterization of cyclin L1 And L2 interactions with CDK11 and splicing factors: influence of cyclin L isoforms on splice site selection. Journal of Biological Chemistry. 283(21).
Muller, D., et al. 2006. Cyclin L1 (CCNL1) gene alterations in human head and neck squamous cell carcinoma. British Journal of Cancer. 94(7).
Sailo, B., et al. 2019. FBXW7 in cancer: what has been unraveled thus far? MDPI Cancers. 11(246).
Toledo, L. I. et al A cell-based screen identifies ATR inhibitors with synthetic lethal properties for cancer-associated mutations, Nat. Struct. Mol. Biol. 18, 721-727, 2011
Yeh, C H., et al. 2016. Oncogenic mutations in the FBXW7 gene of adult T-cell leukemia patients. Proceedings of the National Academy of Sciences. 113(24).
Yeh, C H., et al. 2018. FBXW7: a critical tumor suppressor of human cancers. Molecular Cancer. 17(115).

1. A method of selecting a patient afflicted with a cancer likely to benefit from a CDK11 inhibitor and/or ATR inhibitor treatment, the method comprising:
testing a biological sample obtained from the patient for i) loss of function of F box WD-repeat containing protein (FBXW7), optionally for one or more deleterious mutations in FBXW7 substrate binding domain or a deep chromosomal deletion and/or ii) upregulated CCNL1, optionally a gain or amplification of cyclin L1 (CCNL1); and
selecting the patient having i) loss of function of FBXW7, optionally one or more deleterious mutation in the FBWX7 substrate-binding domain or ii) upregulated CCNL1, optionally amplification of CCNL1, as likely to benefit and/or for treatment with the CDK11 inhibitor and/or the ATR inhibitor.

2. The method of claim 1, wherein the cancer is selected from a cancer listed in Table 2 or Table 3.

3. The method of any one of claim 1 or 2, wherein the cancer is a squamous origin cancer.

4. The method of any one of claims 1 to 3, wherein the cancer is cervical cancer, endometrial cancer, and/or head and neck cancer, optionally wherein the endometrial cancer is uterine carcinosarcoma.

5. The method of any one of claims 1 to 4, wherein the patient is likely to benefit from and/or is selected for treatment with the CDK11 inhibitor.

6. The method of any one of claims 1 to 5, wherein the CDK11 inhibitor is selected from OTS964 or analogs thereof, optionally, OTS964 and analogs thereof, optionally, (R)-1-(4-(1-aminopropan-2-yl)phenyl)-2-hydroxy-4-methylphenanthridin-6(5H)-one hydrochloride or (R)-1-(4-(1-aminopropan-2-yl)phenyl)-2,7-dihydroxy-4-methylphenanthridin-6(5H)-one hydrochloride.

7. The method of any one of claims 1 to 6, wherein the CDK11 inhibitor is OTS964.

8. The method of any one of claims 1 to 4, wherein the patient is likely to benefit from and/or is selected for treatment with the ATR inhibitor.

9. The method of claim 8, wherein the ATR inhibitor is AZD6738, AZ20, BAY1895344, berzosertib, RP-3500, VE-821, VE-822, and/or ETP46464.

10. The method of any one of claims 1 to 9, wherein the biological sample is a tumor sample, optionally a biopsy, or a liquid biopsy comprising circulating tumor cells or circulating tumor DNA.

11. The method of any one of claims 1 to 10, wherein the biological sample comprises cancer cell nucleic acids.

12. The method of any one of claims 1 to 11, wherein the biological sample comprises a protein fraction.

13. The method of any one of claims 1 to 12, wherein the testing comprises assaying for one or more deleterious mutations in the FBXW7.

14. The method of any one of claims 1 to 13, wherein the testing comprises assaying for one or more deleterious mutations in the FBXW7 substrate-binding domain.

15. The method of any one of claims 1 to 14, wherein the testing comprises sequencing a FBXW7 transcript or part thereof.

16. The method of any one of claims 1 to 15, wherein the testing further comprises comparing the FBXW7 transcript sequence to wild type FBXW7 to identify the presence or absence of one or more deleterious mutations.

17. The method of any one of claims 1 to 12, wherein the testing comprises measuring cellular levels of CCNL1 protein or mRNA.

18. The method of claim 17, wherein the cellular levels of the CCNL1 mRNA are measured using RT-PCR or qPCR methods.

19. The method of claim 17, wherein the cellular levels of the CCNL1 protein are measured using a standard polypeptide assay, or by immunohistochemistry of a tumor sample or by immunohistochemistry of a cell sample.

20. The method any one of claims 1 to 19, wherein the method comprises testing the sample for loss of function of F box WD-repeat containing protein (FBXW7), optionally for one or more deleterious mutations in FBXW7 substrate binding domain or a deep chromosomal deletion and upregulated CCNL1, optionally a gain or amplification of cyclin L1 (CCNL1)

21. The method of any one of claims 1 to 16 and 20, wherein the one or more deleterious mutation assayed is any of a point mutation, truncation, or deletion, or combinations thereof.

22. The method of claim 21, wherein the one or more deleterious mutation assayed is one or more of the mutations listed in Table 1.

23. The method of claim any one of claims 1 to 22, wherein the patient is tested for one or more mutations known to be associated with the patient's cancer.

24. The method of any one of claims 1 to 23, wherein the one or more deleterious mutation assayed encodes a R505 mutation.

25. The method of claim 24, wherein the R505 mutation is R505C or R505L.

26. The method of any one of claims 1 to 25, wherein the one or more deleterious mutations results in an increase in the cellular levels of CCNL1 proteins or mRNA relative to cellular levels of CCNL1 in a cell without the one or more deleterious mutations.

27. The method of any one of claims 1 to 23, wherein the one or more deleterious mutations results in an increase in activity of CDK11 relative to activity of CDK11 in a cell without the deleterious mutation.

28. The method of any one of claims 1 to 27, wherein testing for upregulation of CCNL1 comprises assessing for amplification or gain of CCNL1 or a mutant CCNL1.

29. The method of any one of claims 1 to 28, wherein the testing for upregulation of CCNL1 comprises assessing for amplification and/or gain of CCNL1.

30. The method of claim 25, wherein testing for upregulation of CCNL1 comprises assessing for a mutant CCNL1.

31. The method of any one of claims 25 to 30, wherein the assessing for amplification and/or gain of CCNL1 or mutant CCNL1 comprises using qPCR, RNAseq, and/or FISH.

32. The method of any one of claims 1 to 31, further comprising treating the patient with an effective amount of the CDK11 inhibitor treatment.

33. The method of claim 32, wherein the CDK 11 inhibitor is OTS964.

34. The method of any one of claims 1 to 31, further comprising treating the patient with an effective amount of the ATR inhibitor treatment.

35. The method of claim 34, wherein the ATR inhibitor is AZD6738.

36. A kit comprising one or more reagents for performing an assay as described in claims 1 to 35.

37. Use of the kit of claim 36 for selecting and/or treating a patient afflicted with a cancer likely to benefit from a CDK11 inhibitor and/or ATR inhibitor treatment.

38. A method of treating a patient afflicted with a cancer having i) one or more deleterious mutations in F box WD-repeat containing protein (FBWX7), optionally in the FBXW7 substrate binding domain and/or ii) upregulated CCNL1, optionally a gain or amplification of CCNL1, the method comprising administering to said patient a CDK11 inhibitor and/or ATR inhibitor treatment.

39. The method of claim 38, wherein the cancer is selected from a cancer listed in Table 2 or Table 3.

40. The method of any one of claim 38 or 39, wherein the cancer is a squamous origin cancer.

41. The method of any one of claims 38 to 40, wherein the cancer is cervical cancer, endometrial cancer, and/or head and neck cancer.

42. The method of any one of claims 38 to 41, wherein the CDK11 inhibitor is selected from OTS964 and analogs thereof, optionally, (R)-1-(4-(1-aminopropan-2-yl)phenyl)-2-hydroxy-4-methylphenanthridin-6(5H)-one hydrochloride or (R)-1-(4-(1-aminopropan-2-yl)phenyl)-2,7-dihydroxy-4-methylphenanthridin-6(5H)-one hydrochloride.

43. The method of any one of claims 38 to 42, wherein the CDK11 inhibitor is OTS964.

44. The method of any one of claims 38 to 43, wherein the ATR inhibitor is AZD6738.

45. The method of any one of claims 38 to 44, wherein the cancer has one or more deleterious mutations in the FBXW7.

46. The method of any one of claims 38 to 45, wherein the cancer has one or more deleterious mutations in the FBXW7 substrate-binding domain.

47. The method of any one of claims 38 to 46, wherein the one or more deleterious mutations is any of a point mutation, truncation, or deletion, or combinations thereof.

48. The method of any one of claims 38 to 47, wherein the one or more deleterious mutations is one or more of the mutations listed in Table 1.

49. The method of any one of claims 38 to 48, wherein the one or more deleterious mutation encodes a R505 mutation.

50. The method of any one of claims 38 to 49, wherein the R505 deleterious mutation is R505C or R505L.

51. The method of any one of claims 38 to 49, wherein the one or more deleterious mutations results in an increase in the cellular levels of CCNL1 proteins or mRNA relative to cellular levels of CCNL1 in a cell without the one or more deleterious mutations.

52. The method of any one of claims 38 to 51, wherein the upregulation of CCNL1 assayed for is amplification or gain of CCNL1.

53. A method of treating a patient afflicted with a cancer, the method comprising:
obtaining a biological sample;
testing the biological sample for i) one or more deleterious mutations in F box WD-repeat containing protein (FBWX7) substrate binding domain or ii) upregulated CCNL1, optionally a gain or amplification of CCNL1; and
treating the patient having i) a deleterious mutations in F box WD-repeat containing protein (FBWX7) optionally in the substrate binding domain or ii) upregulated CCNL1, optionally a gain or amplification of CCNL1, with a CDK11 inhibitor and/or ATR inhibitor.

54. The method of 53, wherein the cancer is selected from a cancer listed in Table 2 and/or Table 3.

55. The method of any one of claim 53 or 54, wherein the cancer is a squamous origin cancer.

56. The method of any one of claims 53 to 55, wherein the cancer is cervical cancer, endometrial cancer, and/or head and neck cancer, optionally uterine carcinosarcoma.

57. The method of any one of claims 53 to 56, wherein the CDK11 inhibitor is selected from OTS964 and analogs thereof, optionally, (R)-1-(4-(1-aminopropan-2-yl)phenyl)-2-hydroxy-4-methylphenanthridin-6(5H)-one hydrochloride or (R)-1-(4-(1-aminopropan-2-yl)phenyl)-2,7-dihydroxy-4-methylphenanthridin-6(5H)-one hydrochloride.

58. The method of any one of claims 53 to 57, wherein the CDK11 inhibitor is OTS964.

59. The method of any one of claims 53 to 58, wherein the ATR inhibitor is AZD6738.

60. The method of any one of claims 53 to 59, wherein the biological sample is a tumor sample, optionally a biopsy, or a liquid biopsy comprising circulating tumor cells or circulating tumor DNA.

61. The method of any one of claims 53 to 560, wherein the biological sample comprises cancer cell nucleic acids.

62. The method of any one of claims 53 to 61, wherein the biological sample comprises a cancer cell proteins.

63. The method of any one of claims 53 to 62, wherein the testing comprises assaying for one or more deleterious mutations in the FBXW7.

64. The method of any one of claims 53 to 63, wherein the testing comprises assaying one or more deleterious mutations in the FBXW7-binding domain.

65. The method of any one of claims 53 to 64, wherein the testing comprises sequencing a FBXW7 transcript or part thereof, optionally the part corresponding to the substrate binding domain.

66. The method of any one of claims 53 to 65, wherein the testing comprises comparing a FBXW7 sequence to a wild type FBXW7 sequence.

67. The method of any one of claims 53 to 62, wherein the testing comprises measuring cellular levels of CCNL1 protein or mRNA.

68. The method of claim 67, wherein the cellular level of the CCNL1 transcript is detected by RT-PCR method.

69. The method of claim 67, wherein the cellular level of the CCNL1 protein is detected using a standard polypeptide assay or by immunohistochemistry of a tumor sample or by immunohistochemistry of a cell sample.

70. The method of any one of claims 53 to 66, wherein the one or more deleterious mutation assayed are any of a point mutation, truncation, or deletion, or combination thereof.

71. The method of any one of claim 53 to 66, or 70, wherein the one or more deleterious mutations assayed are one or more of the mutations listed in Table 1.

72. The method of any one of claims 53 to 66, or 70 to 71, wherein one or more deleterious mutations assayed are known to be associated with the patient's cancer.

73. The method of any one of claims 53 to 66, or 70 to 72, wherein the one or more deleterious mutation assayed encodes a R505 mutation.

74. The method of any one of claims 53 to 66, or 70 to 73, wherein the R505 mutation is R505C or R505L.

75. The method of any one of claims 53 to 66, or 70 to 74, wherein the one or more deleterious mutations results in an increase in the cellular levels of CCNL1 proteins or mRNA relative to cellular levels of CCNL1 in a cell without the one or more deleterious mutation.

76. The method of any one of claims 53 to 66, or 70 to 75, wherein the upregulation of CCNL1 assayed for is amplification or gain of CCNL1.

77. A package comprising a vial comprising a CDK11 inhibitor and/or ATR inhibitor and a label or instructions, for administering to a patient with a FBWX7 deleterious mutation or upregulation of CCNL1, optionally for use in a method described herein.
